(19) United States
(12) Patent Application Publication
(10) Pub. No.: US 2005/0192585 A1
(43) Pub. Date: Sep. 1, 2005

(54) Surgical saw collet with closed drive ring

(75) Inventor: Bryan D. Simmons, Flower Mound, TX (US)

Correspondence Address: HAYNES AND BOONE, LLP, 901 MAIN ST, SUITE 3100, DALLAS, TX, 75202, US

(73) Assignee: Medtronic, Inc., Minneapolis, MN (US)

(21) Appl. No.: 10/788,957

(22) Filed: Feb. 27, 2004

(51) Int. Cl. A61B 17/32

(52) U.S. Cl. 606/82

(57) ABSTRACT

A surgical instrument includes a motor assembly, a collet assembly connected to the motor assembly, and a dissection tool such as a bone saw blade. The collet assembly includes a body portion with a plurality of engaging members. The dissection tool includes a tool body with a hub positioned in an opening formed within the tool body. The hub includes a plurality of indentions configured to selectively engage with the engaging members.

### FIELD OF THE INVENTION

The present invention relates generally to surgical instruments. More particularly, the present invention relates to coupling arrangements for surgical instruments.

### BACKGROUND

In various surgical procedures, it is necessary to dissect bone or other tissues. In some instances, it may be necessary to cut, grind, shape or otherwise remove hardened materials to make them ready for implantation or to remove them from a patient. Many conventional surgical instruments used for these purposes employ pneumatic or electrical motors to move or rotate a dissection “tool.” In their most basic form, such a surgical instrument comprises a motor portion, a dissection tool having a cutting or abrading element, and a coupling arrangement for connecting the dissection tool to a spindle or collet of the motor. The spindle or collet of the motor is usually housed within a base that is attached to the motor.

While currently known dissection tools, including replaceable dissection tools, offer advantages over earlier designs, it remains desirable to further advance the pertinent art. For example, during a surgical procedure, a dissection tool may oscillate at high speeds, for example approximately 70,000 rpm, and it may not be effectively retained with previously available coupling arrangements under all operating conditions. Also, many dissection tools are inherently weak at their connection to the coupling arrangement.

### SUMMARY

The present disclosure provides an improved surgical instrument, an improved dissection tool, and an improved coupling system for securing the dissection tool, and methods of assembly and using each.

A surgical instrument according to one embodiment of the present invention includes a motor assembly, a collet assembly connected to the motor assembly, and a dissection tool such as a bone saw blade. The collet assembly includes a body portion with a plurality of engaging members. The dissection tool includes a tool body with a hub positioned in an opening formed within the tool body. The hub includes a plurality of indentions configured to selectively engage with the engaging members.

An advantage of one or mole embodiments of the above-described surgical instrument is that a dissection tool can be easily and selectively attached to the collet assembly in a very strong and secure manner.

In another embodiment, a bone saw blade is provided for use with a powered surgical instrument having a collet assembly. The bone saw blade includes a flat extending member having a cutting surface and hub disposed thereon. The hub forms a surrounded opening having a plurality of engagement locations for selectively engaging with corresponding engagement members on the collet assembly. In some embodiments, the surrounded opening is circular in shape.

An advantage of one or more embodiments of the above described bone saw blade is that the bone saw blade can be positioned in multiple locations on the collet assembly, and has improved strength and rigidity.

In another embodiment, a coupling assembly is provided for use with a motor in a powered surgical instrument. The coupling assembly is for selectively attaching a dissection tool, such as a saw blade, to the surgical instrument. The coupling assembly includes a translation member connectable to the motor for receiving a first movement force from the motor and translating it to a second movement force suitable for driving the dssection tool. The coupling assembly also includes a body portion connected to the translation member, a plurality of engagement members, and a selectively engageable plunger. The plunger is configured to be movable into a first position to engage the engagement members with the dissection tool to thereby secure the dissection tool to the collet, and a second position to allow the dissection tool to be separated from the collet. In some embodiments, the plunger and engagement members are configured to be positioned inside an opening of the dissection tool.

An advantage of one or more embodiments of the above-described coupling assemblies is that a dissection tool can be positioned in multiple locations on the collet assembly, and the collet assembly more securely engages with the dissection tool.

Additional advantages and features of the present invention will become apparent from the following description and appended claims, taken in conjunction with the accompanying drawings.

Further areas of applicability of the present invention will become apparent from the detailed description provided hereinafter. It should be understood that the detailed description and specific examples, while indicating the embodiments of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 illustrates a human patient for receiving a surgical procedure associated with one or more embodiments of the present invention.

FIG. 2 illustrates a location of the human patient of FIG. 1 in which bone or tissue is cut or otherwise dissected.

FIG. 3 illustrates a perspective view of a surgical instrument according to one embodiment of the present invention for performing the surgical procedure on the patient of FIG. 1.

FIG. 4 illustrates a top view of the surgical instrument of FIG. 3.

FIG. 5 illustrates a side view of the surgical instrument of FIG. 3.

FIG. 6 illustrates a sectional side view of the surgical instrument of FIG. 3.

FIG. 7 illustrates an exploded view of a collet assembly and dissection tool of the surgical instrument of FIG. 3.

FIG. 8A illustrates a partial cross-sectional side view of the collet assembly of FIG. 7 in a first state.

FIG. 8B illustrates a partial cross-sectional side view of the collet assembly and surgical instrument of FIG. 7 in a second state.

FIG. 8C illustrates a partial cross-sectional side view of the collet assembly and surgical instrument of FIG. 7 in the first state.

FIG. 9A illustrates an enlarged portion of the cross-sectional side view of the coupling assembly of FIG. 8A.

FIG. 9B illustrates an enlarged portion of the cross-sectional side view of the coupling assembly and surgical instrument of FIG. 8B.

FIG. 9C illustrates an enlarged portion of the cross-sectional side view of the coupling assembly and surgical instrument of FIG. 8C.

### DETAILED DESCRIPTIONS

The present invention provides an improved surgical instrument, an improved dissection tool, and an improved coupling system for securing the cutting member, and methods of assembly and using each.

For the purposes of promoting an understanding of the principles of the invention, references will now be made to the embodiments, or examples, illustrated in the drawings, and specific languages will be used to describe the same. It will nevertheless be understood that discussions of one or more specific examples and repetitions of one or more reference numerals is provided for the sake of clarity, and should not limit the scope of the invention. Any alterations and further modifications in the described embodiments, and any further applications of the principles of the invention as described herein are contemplated as would normally occur to one skilled in the art to which the invention relates.

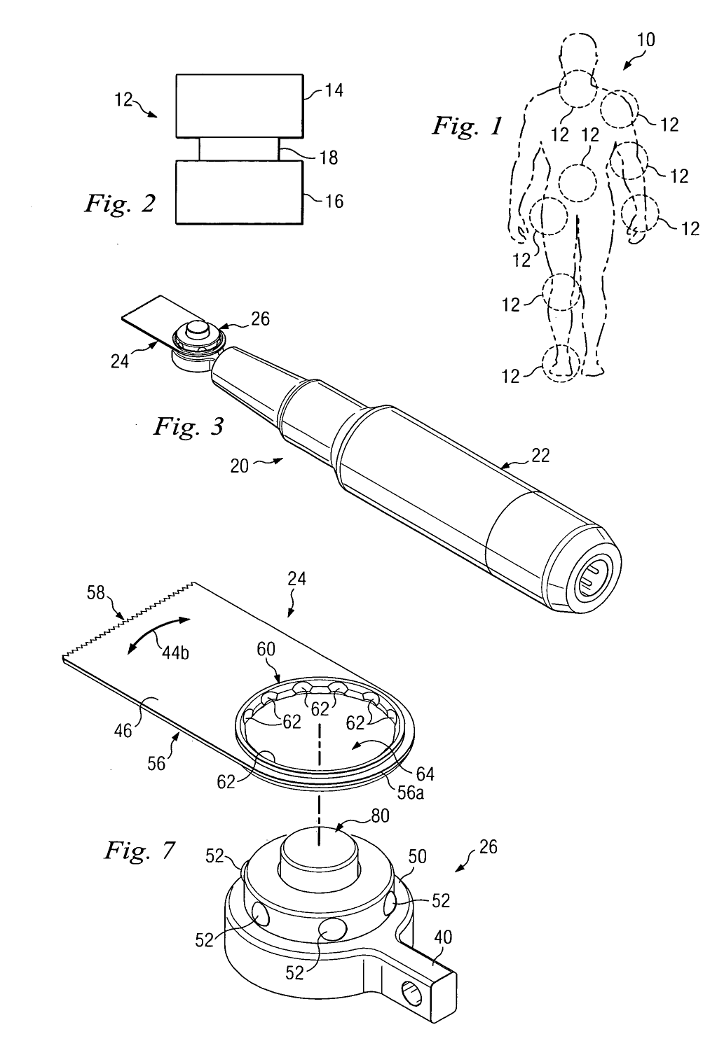

Referring now to FIG. 1, the numeral 10 refers to a human anatomy having one or more bone or tissue locations 12 which may be damaged by injury or disease. As such, it may be desirable to perform a surgical operation on the bone or tissue at one or more of the locations 12, such surgical operation including cutting bone or tissue with a surgical instrument. It will become apparent to those skilled in the art that the described instrument is not limited to any particular surgical operation but has utility for various applications in which it is desired to dissect bone or other tissue. Additional applications include:
1. Arthroscopy—Orthopaedic
2. Endoscopic—Gastroenterology, Urology, Soft Tissue
3. Neurosurgery—Cranial, Spine, and Otology
4. Small Bone—Orthopaedic, Oral-Maxiofacial, Ortho-Spine, and Otology
5. Cardio Thoracic—Small Bone Sub-Segment
6. Large Bone—Total Joint and Trauma
7. Dental.

Referring now to FIG. 2, in a typical surgical procedure, all or a portion of a bone or tissue may need to be cut and/or removed. For example, a bone at location 12 can be cut or dissected at a position 18, thereby creating bone portions 14 and 16. In another example, a ligament 14 can be separated from a bone 16 by cutting or dissecting at the position 18. Such cutting or dissecting can be performed by a surgical instrument discussed in greater detail below.

With reference to FIGS. 3-6, a surgical instrument 20 is illustrated to generally include a motor assembly 22 connected to a dissection tool 24 via a collet assembly 26. In a preferred embodiment, the dissection tool 24 is a cutting tool, particularly a reciprocating saw blade, although other types of tools can also benefit from the present invention. In the exemplary embodiments that will be described, the surgical instrument 20 is electrically powered. It is further understood, however, that many of the teachings discussed herein will have equal application for other surgical instruments, including a pneumatically powered surgical instrument.

As shown in FIG. 6, the motor assembly 22 includes a housing 30 for encasing an electric motor 32. The electric motor 32 is used to turn a spindle 34, which further rotates a first translation member 36. In the present example, the first translation member 36 is an eccentric flywheel rotatable within the housing 30. The first translation member 36 is used to oscillate a shaft 38, which further translates to the tool 24, a saw blade in this example, through the collet assembly 26. In the present embodiment, the shaft 38 is connected to the flywheel 36 in a spherical cavity 39 having a reduced-friction surface, such as via one or more ball bearings.

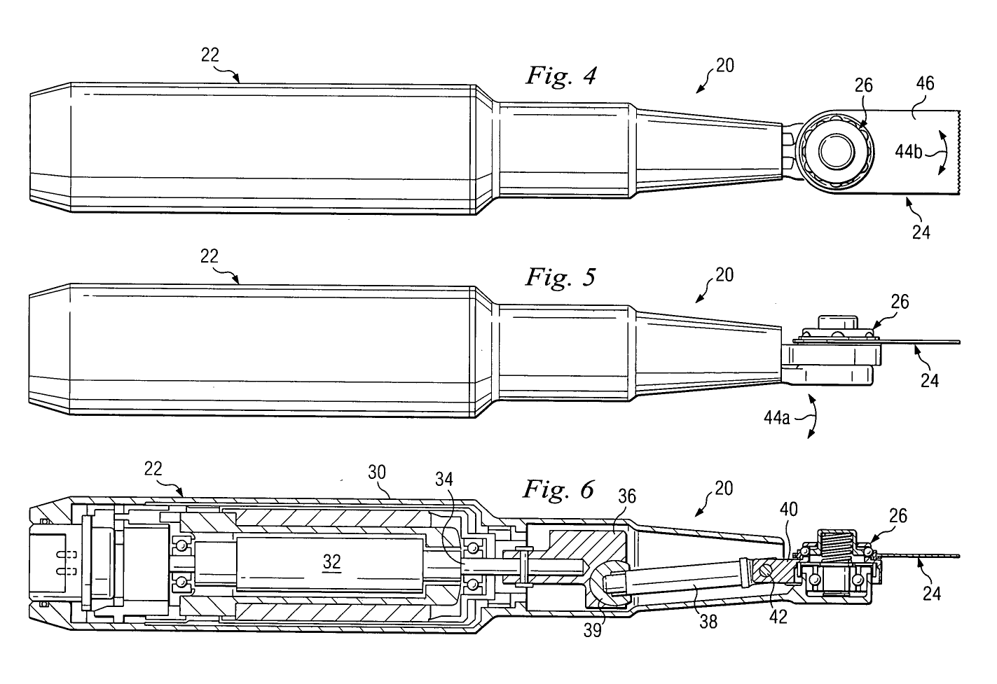

The collet assembly 26 includes a drive member 40 for attaching to the shaft 38 through a second translation member 42. In the present example, the second translation member 42 is a rotatable pin that provides translation between the drive member 40 and the shaft 38 in a vertical plane 44a (FIG. 5), but not in a horizontal plane 44b (FIG. 4). It is noted that in the present example, the plane 44b is parallel with a planar surface 46 of the saw blade 24. Since translation is prevented in the horizontal plane 44b, the drive member 40 rotates back and forth in the plane 44b.

Referring now to FIG. 7, the collet assembly 26 includes a body portion 50 connected to (or integral with) the drive member 40. In other embodiments, the drive member 40 may be a recess in the body portion for receiving the shaft 38. The body portion includes a plurality of engaging members 52. Continuing with the present example, the engaging members 52 are ball bearings that can move back and forth within the body portion 50, as discussed in greater detail below. In other embodiments, the engaging members 52 can be pointed protrusions, cylindrical rods, or many different shapes or combinations of shapes. Still other embodiments may have a continuous surface as its engaging member.

The dissection tool 24, a saw blade in the present example, includes a relatively flat tool body 56 having a plurality of teeth 58 on a distal end and a hub 60 on a portal end. In the present embodiment, the tool body 56 is made of metal, such as titanium or stainless steel. Other examples include carbide, diamond, and combinations thereof. The teeth 58 may be chosen for a particular surgical application (or several applications), and are arranged to promote cutting while moving back and forth in the plane 44b.

The hub 60 is positioned in an opening formed within the tool body 56. The hub 60 may be made of the same material as the tool body 56, or may be formed of a different material. In one embodiment, both the tool body 56 and the hub 60 are made of metal, which are joined together by weld, epoxy, or mechanical force (e.g., the hub is compressed to frictionally engage with the tool body). In another embodiment, the tool body 56 and the hub 60 may be a single monolithic structure.

The hub 60 includes a plurality of indentions 62. The indentions are configured to engage with the engaging members 52 to secure the hub 60 to the engaging members, and thus the body portion 50. In this way, rotational force provided by the tool body 56 is translated to the hub 60, and further to the saw blade 24. In the present embodiment, the engaging members (balls in the present example) 52 also secure the saw blade 24 from being separated from the body portion 50. In other embodiments, a separate member can be used to secure the saw blade 24 to the body portion 50.

In the present embodiment, the hub 60 produces a circular opening 64 within the tool body 56. Further to the present embodiment, a portion 56a of the tool body extends all the way around the opening 64. In other embodiments, the tool body 56a may only extend around a portion of the opening. In these embodiments, the “gap” in the circumferential tool body portion 56a can be used to facilitate the insertion of the hub 60 into the opening when manufacturing the saw blade 24. Also in other embodiments, the opening 64 may be triangular, hexagonal, octagonal, or other shapes, as desired. Furthermore, the opening formed by the tool body portion 56a may be of a different shape than the opening 64. For example, if the opening in the tool body portion 56a were octagonal, then the hub 60, which would also have an octagonal outer shape, would fit in a predetermined arrangement with the tool body 56. The opening 64 could still be circular, or any other desired shape. Such as embodiment can serve to position the indentions 62 at an exact location. For further example, if there were eight indentions 62, each indention could be positioned in the hub 60 at a predetermined place of the octagonal outer shape. The octagonal shape can also provide mechanical strength to the joint between the hub 60 and the tool body 56.

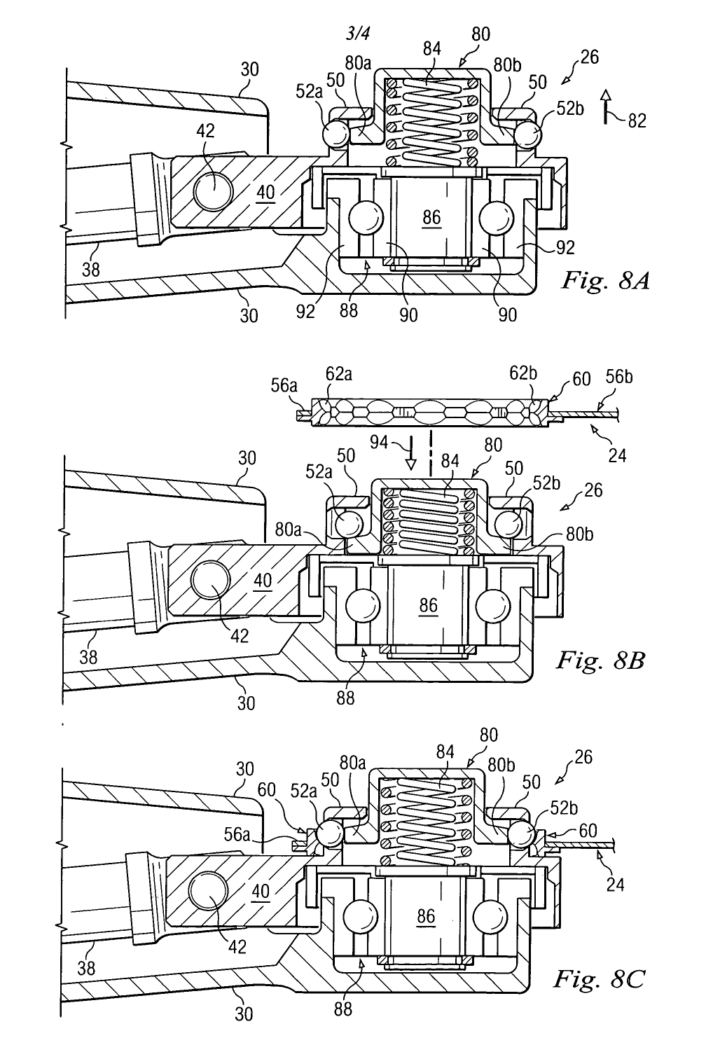

Referring to FIGS. 8A-8C, the collet assembly 26 includes a plunger 80 that is continually urged in a direction 82 by a compression device 84. In the present embodiment, the compression device 84 is a coil spring, alternatives include a leaf spring, a pressurized fluid, and other compressible material. The coil spring 84 presses against a post 86 to cause the plunger 80 to move in the direction 82. The collet assembly 26 also includes a ball bearing assembly 88, including an inner race 90 and outer race 92, which supports the oscillation of the collet assembly, and thus the saw blade 24.

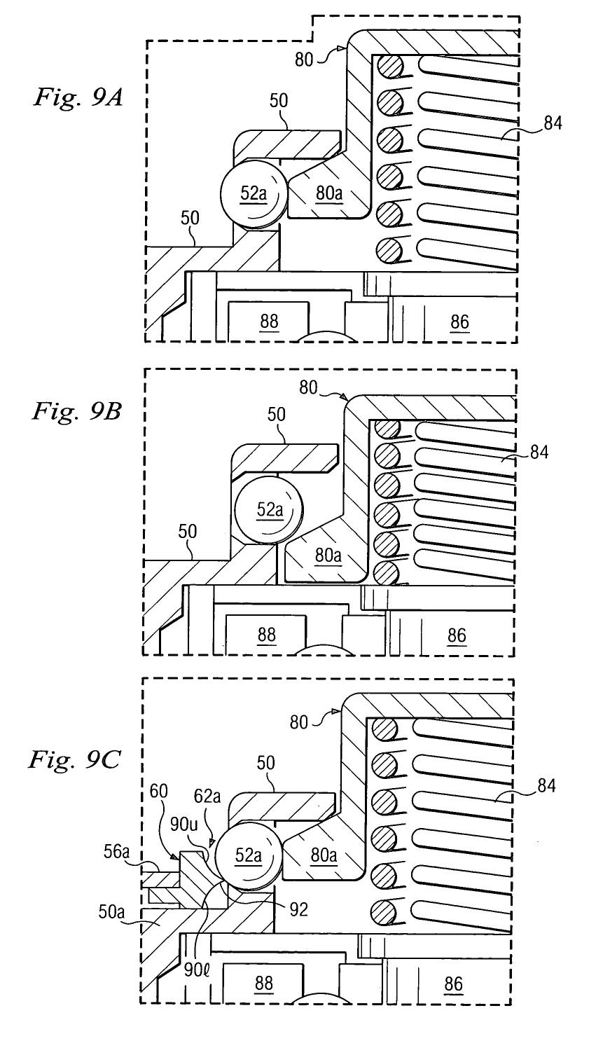

Referring specifically to FIG. 8A and also to FIG. 9A, in a first state, the coil spring 84 presses the plunger 80 in the direction 82 so that a portion of the plunger (e.g., portions 80a and 80b as shown in FIG. 8A) positions or presses against the engaging members (balls 52a and 52b, respectively) in an extended position.

Referring now to FIG. 8B and FIG. 9B, when it is desired to insert or remove the dissection tool 24 into the collet assembly 26, a force 94 is applied to the plunger 80, such as by a person's finger. Continuing with the present example, this places the collet assembly 26 into a second state where the balls 52a, 52b are not being positioned or pressed against by the plunger portions 80a, 80b, respectively. As a result, the saw blade 24 can be lowered onto the collet assembly 26 and the balls 52a, 52b will move in towards a central axis of the collet assembly to further receive the hub 60 of the saw blade.

Referring now to FIG. 8C and FIG. 9C, once the dissection tool 24 is in a desired location, the collet assembly 26 can be returned to the first state. As a result, the balls 52a, 52b (in the present example) are positioned and pressed away from the central axis of the collet assembly 26 so that they engage with respective indentions 62a, 62b of the hub 60. In this way, the saw blade 24 is locked into the collet assembly 26 until the plunger 80 is pressed again to put the collet assembly back into the second state.

Referring specifically to FIG. 9C, in one embodiment, each of the indentions 62 are uniquely configured to allow the dissection tool 24 to be locked into the collet assembly 26 in one alignment or flipped over in the opposite alignment. For example, the indention 62a includes two sub-indentions 90u and 901 and a protrusion 92. When in the locked first state, the protrusion 92 is held below the corresponding ball 52a into frictional engagement with the body portion 50a. As a result movement by the ball 52a, the body portion 50a, or both causes the dissection tool 24 to move as well.

It will be noted that several advantages are provided by one or more of the above-described embodiments. For one, the dissection tool 24 is strengthened by the hub, as compared to prior art dissection tools. Another advantage is that the dissection tool 24 can be connected to the coupling assembly 26 in many different directions, or may be flipped over 180 degrees. Yet another advantage is that the contact between the coupling assembly 26 and the dissection tool is distributed over a significant area. It is understood that some embodiments may not have any of the above-listed advantages, while other embodiments may have combinations of these advantages. Other advantages will also be readily apparent to those of ordinary skill in the art.

While the invention has been particularly shown and described with reference to the preferred embodiment thereof, it will be understood by those skilled in the art that various changes in form and detail may be made therein without departing from the spirit and scope of the invention. For example, while the present illustrative embodiments show electric powered motors, it is contemplated that the improvements described herein may be applied in an equal fashion to other motors, such as ultrasonic motors-run by piezo-electric or magneto-strictive forces. Furthermore, the various means described for connecting various housings or components may be replaced by other suitable means in ways known to those in the art. Therefore, the claims should be interpreted in a broad manner, consistent with the present invention.

1. A surgical instrument comprising:
a motor assembly;
a collet assembly connected to the motor assembly and including a body portion with a plurality of engaging members;
a dissection tool including a tool body with a hub positioned in an opening formed within the tool body, the hub including a plurality of indentions configured to selectively engage with the engaging members.

2. The surgical instrument of claim 1 further comprising:
a housing for encasing the motor and the collet assembly, wherein the collet assembly is capable of rotational movement inside the housing.

3. The surgical instrument of claim 1 wherein the dissection tool is a relatively flat reciprocating saw blade.

4. The surgical instrument of claim 1 wherein the motor is electrically powered.

5. The surgical instrument of claim 4 wherein the electric motor is used to turn a spindle, which further rotates an eccentric flywheel, which further provides an oscillating motion to the collet assembly.

6. The surgical instrument of claim 5 wherein the collet assembly includes a drive member for attaching to a shaft connected to the flywheel.

7. The surgical instrument of claim 1 wherein the hub includes a complete, circular aperture for receiving a portion of the collet assembly.

8. The surgical instrument of claim 7 wherein the opening in the tool body is non-circular.

9. The surgical instrument of claim 7 wherein a portion of the tool body extends all the way around the opening.

10. The surgical instrument of claim 1 wherein the collet assembly includes a plunger in contact with a compression device.

11. The surgical instrument of claim 10 wherein the compression device is a coil spring, the coil spring configured for positioning the plunger in a first state to urge the engaging members into corresponding indentions in the hub and for positioning the plunger in a second state whereby the engaging members are readily separable from their corresponding indentions.

12. The surgical instrument of claim 1 wherein the engaging members are balls.

13. The surgical instrument of claim 1 wherein at least one of the indentions includes two sub-indentions and a protrusion therebetween so that when the engaging members are engaged with the indentions, the hub is locked in place and frictionally engaged with the collet assembly.

14. The surgical instrument of claim 1 wherein the tool body and hub are both made of metal and are joined together by weld, epoxy, or mechanical force.

15. The surgical instrument of claim 1 wherein the tool body and hub are a single monolithic structure.

16. A bone saw blade for use with a powered surgical instrument having a collet assembly, the bone saw blade comprising:
a flat extending member;
a cutting surface disposed on the flat extending member;
a hub disposed on the flat extending member, the hub forming a surrounded-opening having a plurality of engagement locations for selectively engaging with corresponding engagement members on the collet assembly.

17. The bone saw blade of claim 16 wherein the surrounded opening is circular in shape.

18. The bone saw blade of claim 16 wherein the engagement locations are indentions for selectively receiving protrusion from the collet assembly.

19. The bone saw blade of claim 16 wherein a portion of the flat extending member extends completely around the opening.

20. The bone saw blade of claim 16 wherein at least one of the engagement locations includes two sub-indentions and a protrusion therebetween so that when one of the engagement members is engaged with the indentions, the hub is locked in place and frictionally engaged with the collet assembly.

21. The bone saw blade of claim 16 wherein the tool body and hub are both made of metal and are joined together by weld, epoxy, or mechanical force.

22. The bone saw blade of claim 16 wherein the tool body and hub are a single monolithic structure.

23. A coupling assembly for use with a motor in a powered surgical instrument and for selectively attaching a dissection tool to the surgical instrument, the coupling assembly comprising:
a translation member connectable to the motor for receiving a first movement force from the motor and translating it to a second movement suitable for driving the dissection tool;
a body portion connected to the translation member;
a plurality of engagement members;
a selectively engageable plunger configured to move into a first position to move the engagement members to engage with the dissection tool to secure the dissection tool to the collet, and a second position to allow the dissection tool to be separated from the collet.

24. The coupling assembly of claim 23 wherein the plunger and engagement members are configured to be positioned inside an opening of the dissection tool.

25. The coupling assembly of claim 23 wherein the engagement members are spherical shaped and are positioned in equal spaces around the plunger.

26. The coupling assembly of claim 23 further comprising:
a compression device for urging the plunger towards the second position.

27. The coupling assembly of claim 26 further comprising:
a post engaged with the compression device; and
a ball bearing assembly to support the second movement for driving the dissection tool.

28. The coupling assembly of claim 27 wherein the dissection tool is a bone saw.
